(19) United States
(12) Patent Application Publication
(10) Pub. No.: US 2014/0107521 A1
(43) Pub. Date: Apr. 17, 2014

(54) FUNCTIONAL BRAIN CONNECTIVITY AND BACKGROUND NOISE AS BIOMARKERS FOR COGNITIVE IMPAIRMENT AND EPILEPSY

(71) Applicant: Case Western Reserve University, Cleveland, OH (US)

(72) Inventor: Roberto Fernandez Galan, Cleveland, OH (US)

(73) Assignee: Case Western Reserve University, Cleveland, OH (US)

(21) Appl. No.: 14/053,173

(22) Filed: Oct. 14, 2013

Related U.S. Application Data
(60) Provisional application No. 61/713,257, filed on Oct. 12, 2012.

(51) Int. Cl. A61B 5/0476; A61B 5/00

(52) U.S. Cl. CPC A61B 5/0476; A61B 5/4094; A61B 5/4088
USPC 600/544

(57) ABSTRACT

A method of determining functional brain connectivity and its application as a biomarker is presented. Brain activity signals are measured to detect a functional connection between a first region of a brain and a second region of said brain. The direction of the functional connection is determined by determining whether said first region excites or inhibits the second region more strongly than the second region excites or inhibits the first region. The functional connectivity is then used as a biomarker to predict the existence of a condition, such as epilepsy or autism. The functional connection may be measured while said brain is in a resting state.

### CROSS-REFERENCE TO RELATED APPLICATIONS

This application claims priority to U.S. Provisional Patent Application No. 61/713,257 entitled “FUNCTIONAL BRAIN CONNECTIVITY AND BACKGROUND NOISE AS BIOMARKERS FOR COGNITIVE IMPAIRMENT,” filed on Oct. 12, 2012, which is hereby incorporated by reference in its entirety.

### FIELD OF INVENTION

The present invention generally relates to a system and method of using functional brain connectivity as a biomarker for determining cognitive impairment as well as to detect epileptic zones in the brain.

### BACKGROUND

There is a current debate in the autism field related to the concept of “disconnection” in the autistic brain that became popular from psychological and neuroimaging evidence. Proposals of disruption of coordinated timing in neuronal activity in autism were advanced, along with the possibility of reduced brain synchronization, but other suggestions appeared indicating that while the brains of those with autism can be perhaps generally characterized as disconnected, local networks may be more connected. Neuroimaging evidence has traditionally supported the concept of reduced functional connectivity in autism, e.g., under-connectivity has been documented in the baseline resting state of cortical networks. Conversely, evidence indicating enhanced connectivity between brain regions has appeared very recently, suggesting that the “under-connectivity theory of autism” may not suffice to describe the brain coordination dynamics characteristic of this condition. For example, enhanced thalamocortical connectivity in high-functioning autism has been reported, and stronger connectivity between specific cortical areas at rest has also been noted, as well as increased connectivity in striatal regions of children with ASD. These findings emphasize that it may not be a matter of less connectivity in autism, but of a different style of coordination dynamics between specific areas and perhaps also globally.

Most of these studies have relied on metabolic measurements. A complementary approach is the analysis of electroencephalographic signals, which have greater time resolution thus allowing for the study of transient coordination patterns. Indeed, the crucial aspect of these patterns in normal cognition is their transience: widespread long-lasting synchrony is normally associated with unconsciousness or disease. Thus, studies evaluating electroencephalographic or magnetoencephalographic recordings reported evidence for distinct patterns of brain coordinated activity derived from synchronization measures. The documentation of unique coordination patterns can reveal which brain areas operate with different levels of coordination and when these differences occur so that, perchance, therapeutical interventions may target specific brain areas. These investigations may as well contribute to the diagnosis of autism early in development. However, as a note of caution, one should keep in mind that the notion of functional connectivity in the literature encompasses a wide variety of mathematical techniques applied to different recording modalities, and hence a direct comparison between studies may be misleading.

While connectivity measures are providing important insight into brain function, an area that remains very much under-investigated relates to the detailed analysis of the background, resting nervous system activity. Examination of noise and fluctuations in neurophysiological signals rather than concentrating on averages and magnitudes as is customary, is crucial for a complete understanding of nervous system function and its relation to behavior, as sensory stimuli are known to modulate the ongoing neural activity. From a practical perspective, studying ongoing activity is easier than performing cognitive/behavioral tasks in experimental recordings.

Here we present an analysis method that allows one not only to determine functional connectivity in a standardized way, but also to reconstruct the spatio-temporal characteristics of the “noise”, i.e. the random input driving the network in the resting state, which is characterized by a minimal presence of, or attention to, sensory stimulation. The approach is based on recent theoretical work from one of our labs, showing that spontaneous brain activity can be described as a stochastic system driven by Gaussian noise that may be spatially and temporally correlated. Importantly, we find that not only the functional brain connectivity, but also the spatial pattern of stochastic background activity can serve as reliable discriminators between individuals with autism and control participants. This suggests that our model provides an efficient biomarker for Asperger's syndrome, and perhaps more generally, for autism spectrum disorders as well as other cognitive phenotypes.

Approximately one third of patients with epilepsy are intractable to medical therapy and surgery becomes an alternative option. Resective and non-resective surgical strategies have been used to attain seizure freedom in a proportion of these patients. Despite the advances in diagnostic imaging and electrophysiological techniques, the surgical outcome remains modest at best. Only 47-53% patients with temporal lobe epilepsy (TLE) remain seizure free, 10 years after anterior temporal lobectomy. Consequently, the development of better tools to localize the epileptogenic zone and guide resection with greater precision is of paramount importance.

The epileptogenic zone is a theoretical concept and is defined as the area of cortex that is indispensable for the generation of epileptic seizures, which on total removal or disconnection renders the patient seizure free. While this idea is still valid and practical, the concept of epilepsy as a localized region of abnormality has since evolved to a disease of cortical networks with nodes and connections involving even regions farther from the seizure onset zone. Altered perturbations and interactions between various nodes in multiple cortical networks in epilepsy have been intensively studied in the past few years. In order to detect and quantify these interactions, several measures of functional connectivity have been proposed. By “functional”, it is implied that connectivity is operationally defined, according to the tools used to quantify statistical interdependences between simultaneous recordings from different brain areas. A plethora of quantitative methods have been used to analyze functional connectivity with signals from EEG, MEG and fMRI, and in particular, in the context of epileptogenesis, seizure propagation and classification of epilepsy. Altered connectivity in different brain regions has been reported in childhood absence epilepsy, juvenile myoclonic epilepsy, photosensitive epilepsy syndromes and focal epilepsies like TLE.

In this context, previous studies have shown that measures of synchrony in interictal intracranial EEG remain stable over long periods of time and can be used to detect seizure onset zone. Methods like cross-correlation and coherence were applied to EEG signals decades ago to study seizure spread and inter-hemispheric interactions in focal epilepsy. Surgical removal of sharply defined synchronization clusters in electrocorticography from lateral temporal cortex of patients with intractable epilepsy assessed by linear correlation, phase synchronization and mutual information correlated with seizure outcome. In a small sample of nine patients with medically intractable epilepsy studied using subdural grids, Schevon and collaborators found that resection of regions of local hypersynchrony between adjacent pairs electrodes measured by mean phase coherence improved seizure outcome. Similar findings were noted in a recent study by Warren and colleagues who showed that linear cross-correlation and mean phase coherence were high inside the seizure onset zone. Importantly, they noted lower synchrony between seizure generating areas and the surrounding brain regions using the same measures in the local field potential recorded from intracranial EEG. They concluded that in focal epilepsy, the seizure onset zone is functionally isolated from the surrounding brain regions.

In recent computational studies, virtual brains modeling diseased states like epilepsy displayed lower structural complexity compared to models of normal neural function. A measure of neuronal complexity loss estimated from subdural electrodes during presurgical evaluation of epilepsy correctly identified 86.7% of the patients with respect to seizure outcome, consistently with other reports.

Along the lines of these previous studies on correlated neural activity and network complexity, we investigated the utility of normalized cross-correlations (Pearson's correlation coefficient) between SEEG signals as a measure of functional brain connectivity to determine if the strength and heterogeneity of functional connections predict surgical outcome of temporal lobectomy in intractable temporal lobe epilepsy. Cross-correlations provide an intuitive and computationally efficient measure of the global entrainment between two signals, which makes them a natural choice for a pilot study.

No other studies have attempted to shape a surgical outcome measure based on both strength and variability of functional connectivity. We hypothesized that assessing functional connectivity of both mesial and neocortical temporal regions together using SEEG would enhance predictability of our measure.

### SUMMARY

A method of determining functional brain connectivity and its application as a biomarker is presented. Brain activity signals are measured to detect a functional connection between a first region of a brain and a second region of said brain. The direction of the functional connection is determined by determining whether said first region excites or inhibits the second region more strongly than the second region excites or inhibits the first region. The functional connectivity is then used as a biomarker to predict the existence of a condition, such as epilepsy or autism. The functional connection may be measured while said brain is in a resting state.

### BRIEF DESCRIPTION OF THE DRAWINGS

The operation of the invention may be better understood by reference to the detailed description taken in connection with the following illustrations, wherein:

FIG. 1 illustrates sensor distribution and functional connectivity.

FIG. 2 illustrates global properties of the functional connectivity.

FIG. 3 illustrates investigation of pairwise functional connections.

FIG. 4 illustrates changes in functional brain connectivity in ASD relative to control.

FIG. 5 illustrates dominant changes in functional connectivity as an arrow plot.

FIG. 6 illustrates statistical properties of the background noise driving the brain network.

FIG. 7 illustrates a table showing separability indices for the covariance matrices of the noise, Q.

FIG. 8 illustrates a table showing separability indices for the connectivity matrices, W.

FIG. 9 illustrates dominant patterns of background noise driving the brain network.

FIG. 10 illustrates sample traces of SEEG for four patients with amplitude in standard deviation units (z-score) and connectivity matrices for four patients (top to bottom) and their small changes over time (left to right).

FIG. 11 illustrates graphs A, B and C wherein A shows the mean connection strength and the variability of the connections are both significantly lower in patients with a positive outcome; B shows when used in combination, these two parameters can resolve both groups of patients with only 13% error; and C shows the sensitivity, specificity and accuracy of the patient classification are highly significant.

FIG. 12 illustrates coronal sections showing parcellation units of the temporal lobe.

FIG. 13 illustrates connectivity matrices for all patients.

FIG. 14 illustrates averaged connectivity values between areas for positive and negative surgical outcomes.

FIG. 15a illustrates a first portion table showing clinical characteristics of the patient cohort.

FIG. 15b illustrates a second portion table showing clinical characteristics of the patient cohort.

FIG. 15c illustrates a third portion table showing clinical characteristics of the patient cohort.

### DETAILED DESCRIPTION

Reference will now be made in detail to exemplary embodiments of the present invention, examples of which are illustrated in the accompanying drawings. It is to be understood that other embodiments may be utilized and structural and functional changes may be made without departing from the respective scope of the invention. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

The outline of our results is as follows. We first investigate global differences between the connectivity matrices in the control and ASD groups. In particular, we show that the matrices of each group cluster in a high-dimensional space. We then investigate which specific features of the matrices account for this clustering. Specifically, we show that certain pair-wise interactions are significantly different. Finally, we demonstrate that in addition to having some different functional connections, the brains from control and ASD differ in the spatial distribution of background noise driving the network.

### Results

In the absence of stimulation, the non-linear dynamics of the brain reduces to noise-driven fluctuations around a state of equilibrium, which in realistic neural-mass models of brain dynamics corresponds to a hyperbolic fixed point. The presence of background noise does not allow the system to quench at the fixed point but instead the noise perturbs the system in a continuous manner so that fluctuates around the equilibrium. Thus, consistent with the approach used by several authors, large-scale spontaneous brain activity can be described as a linear multivariate stochastic system, which in its continuous version in time is equivalent to an Ornstein-Uhlenbeck process (see Methods)

$$\frac{d {x}_{i}}{d t} = {\sum }_{j = 1}^{N} {W}_{ij} {x}_{j} + {η}_{i} , \qquad ( 1 )$$

where Wij is the functional connectivity matrix, i.e. the coupling between the j-th and the i-th nodes; xi is the neural activity of the i-th node with respect to baseline, measured as the signal from the i-th MEG channel; gi are the residuals (background, uncorrelated white noise) of the i-th channel; and N is the number of nodes (channels). The sign of Wij can be thought of as functional excitation and inhibition, although these do not necessarily represent excitatory and inhibitory synaptic connections at the cellular level. From a physiological perspective Wij can be thought of as the net effect of many excitatory and inhibitory synapses plus other neuro modulators converging onto the area associated with a node. The units of Wij are reciprocal of time, i.e. frequency units.

Wij can be obtained from the empirical data xi(t) with a linear regression to equation (1). The background noise driving the network gi(t) can also be obtained as the residuals of the linear regression. The details of this fitting procedure are provided in Methods. The determination of the connectivity matrix and the background noise are the core of our approach and is what allow us to investigate significant differences between the brains of control subjects and those with ASD.

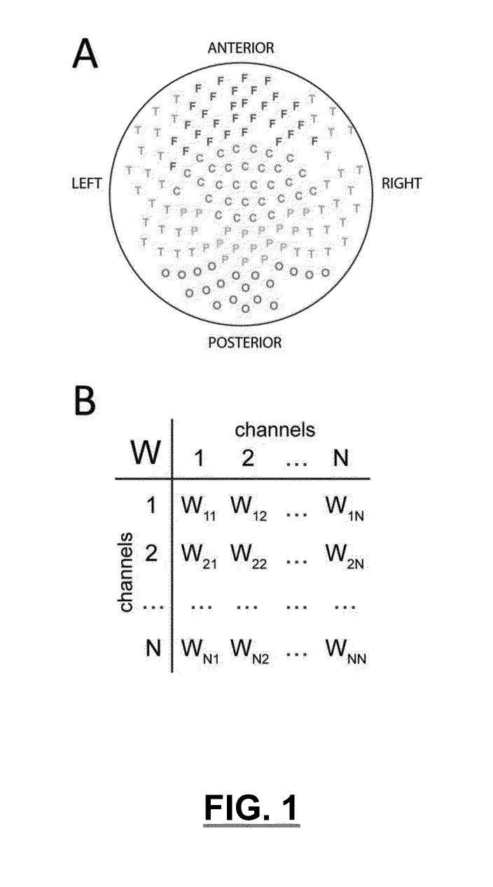

FIG. 1a shows a stereographic projection of the MEG sensors distributed over the scalp onto a planar circle. Although the sensor grid has 151 sensors, we only show the positions of the 141 sensors that were used in all the subjects of our study. The other 10 were discarded because they contained artifacts or very low signal-to-noise ratios in at least one subject, thereby precluding their use in comparative analyses. Thus, the dimensions of the functional brain connectivity matrix for each subject are 141×141. The sensors cover the occipital (O), frontal (F), central (C), parietal (P) and temporal (T) areas. Each ordered pair of sensors (i,j) defines an entry in the connectivity matrix Wij (FIG. 1b) which is obtained from the data using a linear regression to equation (1), as mentioned above.

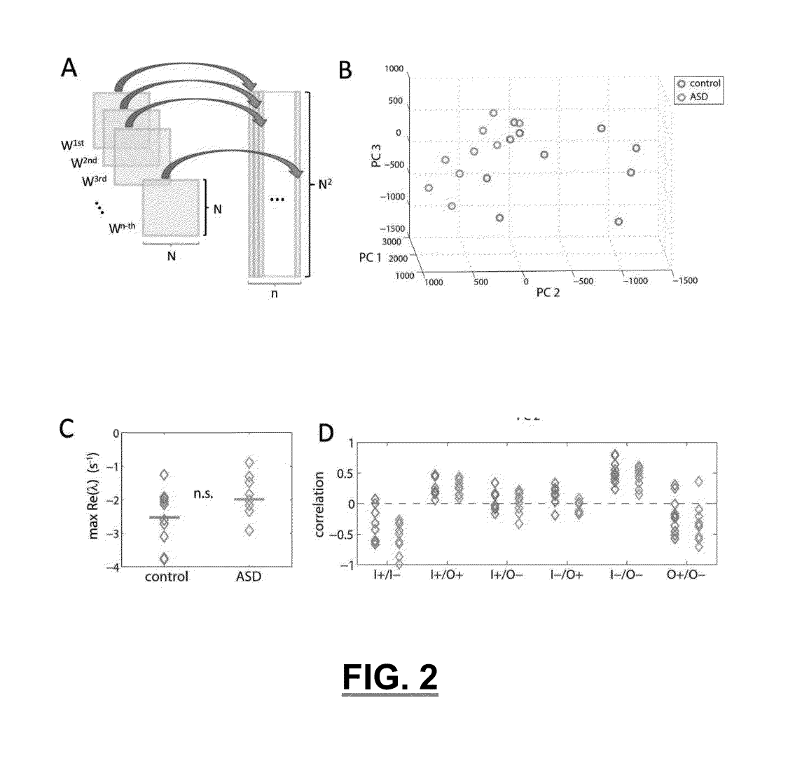

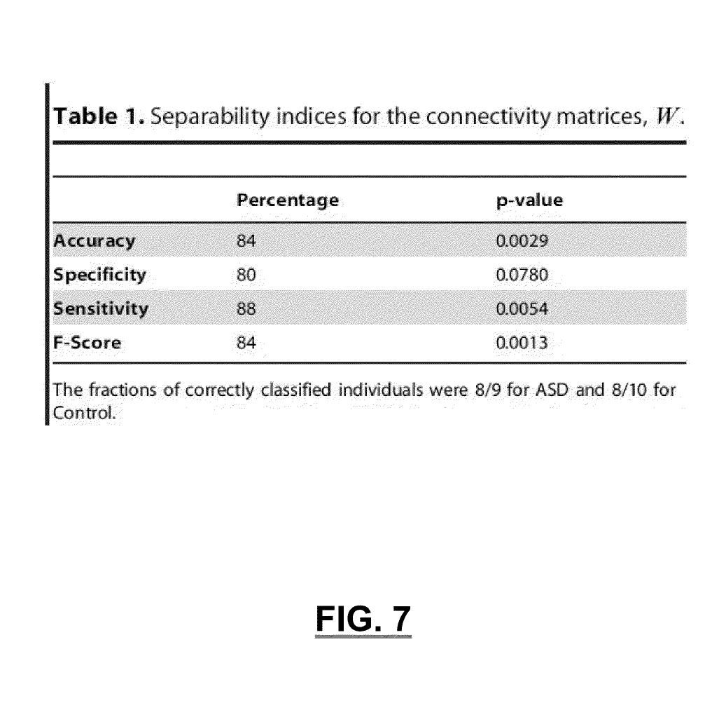

After obtaining the connectivity matrices for the control subjects (n=10) and the ASD subjects (n=9), we investigated if these matrices where significantly different when considered as a whole. To this end, we first “reshaped” the matrices as column vector (FIG. 2A), so that each brain was now represented as a point in a high-dimensional space with 141×141=19,881 dimensions. Then, we investigated the separability of the brains from both groups using a support-vector classifier (see Methods). The accuracy of the discrimination between both groups was 84% (FIG. 7). The good separability of the control and ASD groups can be visualized by looking at the projections of the vectors onto their first three principal components, as displayed in FIG. 2B. Note that even in this low-dimensional projection, both groups can be almost perfectly discriminated.

Having shown that the functional connectivity matrices for control and ASD subjects are different when considered as a whole, we proceeded to investigate if those differences resulted from difference in the global properties of the matrices. We first investigated the maximal real part of the eigenvalues, which corresponds to a linear stability analysis of system (1). In order for (1) to be a valid model of brain dynamics in the resting state, all eigenvalues must have a negative real part. Otherwise, the brain would be linearly unstable, i.e. epileptic. FIG. 2C shows the distribution of the maximal real part of the eigenvalues. There is one data point per matrix corresponding to the eigenvalue with largest real part. The medians of the distributions for the control and ASD cases are not significantly different (p. 0.05; Wilcoxon's ranksum test), indicating that both groups have brains that are equally stable and dissipate perturbations over the same time scale of less than 1 s. We then investigated whether the brains were balanced differently, i.e. whether the distribution of excitation and inhibition across the network nodes was different. A means of keeping a network balanced is by ensuring that each node receives as much excitation as inhibition. This would lead to a high correlation (in absolute value) of the input I+ vector, which is the sum across all rows of the excitatory entries in Wij, with the Ivector, which is the sum across all rows of the inhibitory entries in Wij. This is indeed the case for both the control and ASD groups but there are no significant differences between them (FIG. 2D). Another means of keeping the network balanced is by ensuring that each node provides on average as much excitation as inhibition to other nodes in the network. This would lead to a high correlation (in absolute value) of the output O+ vector, which is the sum across all columns of the excitatory entries in Wij, with the O− vector, which is the sum across all columns of the inhibitory entries in Wij. That is also the case for both the control and ASD groups but again, there are no significant differences between them (FIG. 2D). Neither are significant differences for the rest of the correlations, I+/O+, I+/O2, I2/O+ and I2/O2 (FIG. 2D).

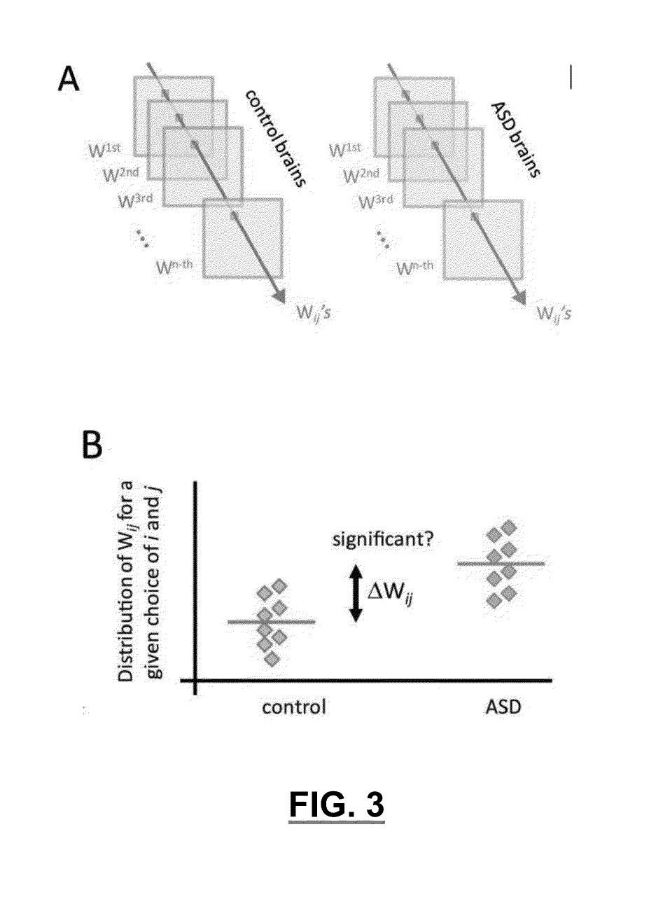

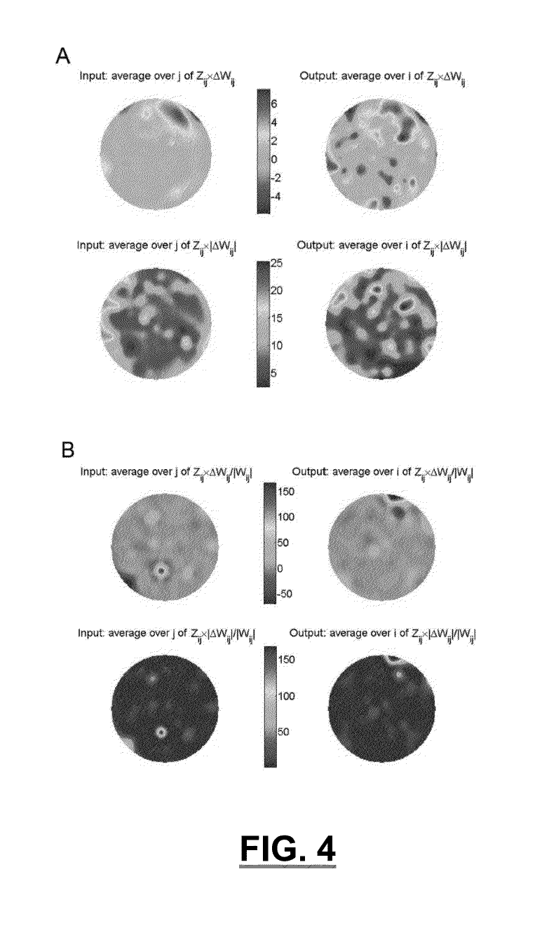

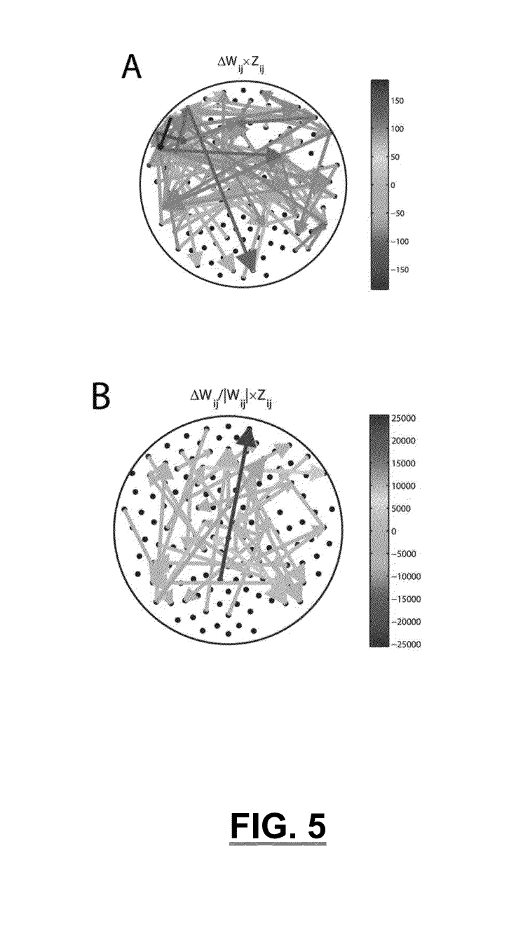

Since global properties could not account for the differences between connectivity matrices, we also investigated all pairwise interactions, Wij and their possible alterations in ASD relative tocontrol (FIG. 3). To this end, for each Wij we collected all the values across the control and ASD subjects (FIG. 3A) and built the distributions of these values for both groups (FIG. 3B). We then asked if the difference of the means was significant using a random permutation test (see Methods). This test also returns the z-score for the actual difference of the means, Zij. We recall that a z-score of 1.5 roughly corresponds to a 95% percentile and hence, larger values indicate a highly significant difference. FIG. 4A shows the actual difference of the means ΔWᵢⱼ≡Wᵢⱼ^{ASD}−Wᵢⱼ^{control} where the Wij are averaged for each group, weighted by the z-score and averaged across all inputs (left) or outputs (right). The most relevant changes appear in nodes located in frontal, parietal, and temporal areas. Probably more informative is the normalization of these changes relative to the magnitude of control the connections in the control case (FIG. 4B), i.e., ΔWᵢⱼ/|Wᵢⱼ^{control}|. This representation clearly shows that the major significant change in functional connectivity occurs between parietal and frontal areas. Another way of visualizing these changes is presented in FIG. 5A, for the largest absolute changes, and 5B for the largest relative changes. By far, the increase in functional excitation between a parietal and a frontal area is the largest relative change in connectivity. All together these results demonstrate that there are specific changes in connectivity in ASD relative to control that account for the separability of the brains from these groups when considered as a whole.

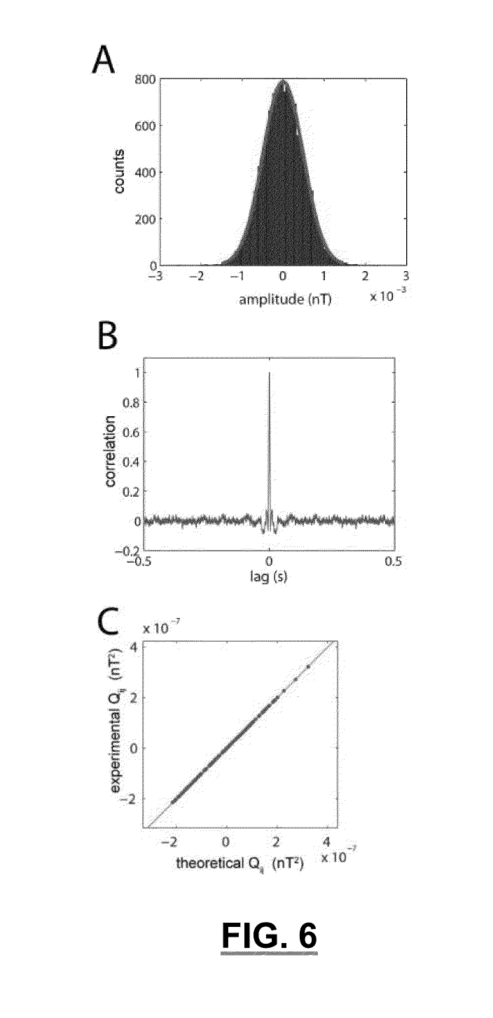

We then asked whether in addition to significant connectivity changes, the background noise driving the brain activity in the resting state could also be different in ASD compared to control. To test this, we obtained the traces of background noise gi(t) as the residuals from the linear regression of the MEG signals to system (1), as described in Methods. We first note that the residuals are normally distributed, as shown in FIG. 6A for the residuals of an arbitrary channel. In addition, the residuals are almost perfectly white, as shown by a sharp centered peak in the autocorrelogram. These results are very important as they provide a validation for model (1). Indeed, if the residuals were neither normally distributed nor white, one would conclude that there is some structure in the data, e.g. a temporal modulation, which cannot be accounted for by a multivariate stochastic linear model. An additional validation for model (1) is provided by the excellent agreement between the covariance matrix of the residuals, Qij, as obtained directly from the residuals, and the Qij obtained analytically from the connectivity matrix, as explained in Methods. This agreement is shown in FIG. 6C for an arbitrary subject as a perfect correlation between the theoretical and experimental values. Altogether, these results demonstrate that model (1) is more than an appropriate and convenient parametric description of the brain dynamics in the resting state: it is the actual form of these dynamics.

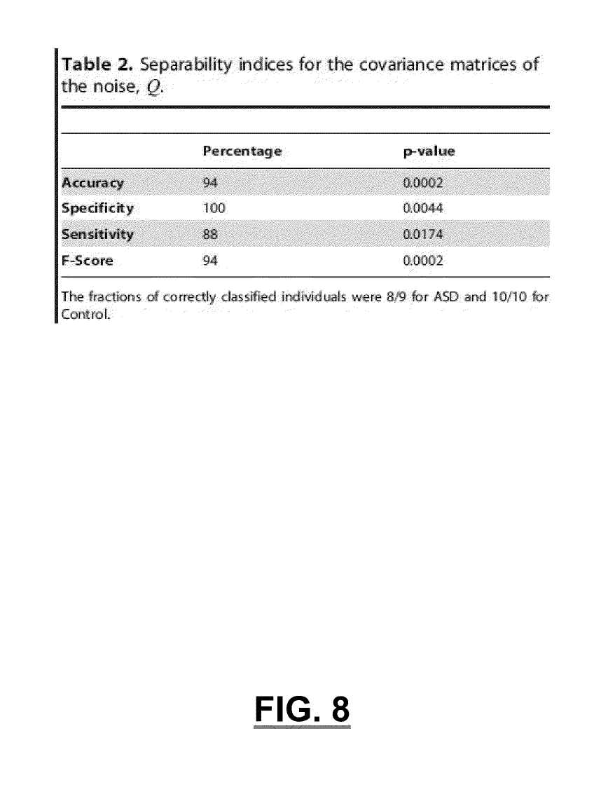

The matrices Q from control and ASD can be well discriminated with a support-vector classifier, as was the case with the connectivity matrices, W above. Interestingly, the discrimination based on the covariance matrix of the noise is even better: 94% accuracy (FIG. 8) compared to the 84% accuracy based on the connectivity matrices (FIG. 7).

The determination of the background noise traces allows us to investigate its spatial structure as well. Indeed, whereas the noise is not temporally correlated (white), it displays spatial correlations.

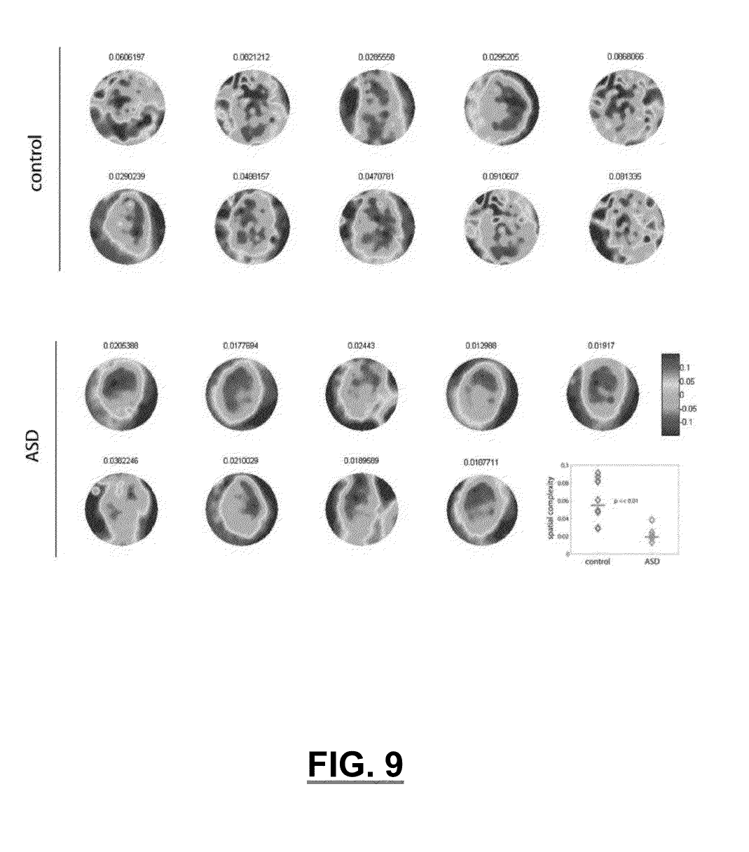

The spatial patterns of the correlated noise are evident from a principal component analysis of the residuals (see Methods). To this end, we first compute the principal components as the eigenvectors of Qij. Then we calculate the weighted average of the eigenvectors with their eigenvalue as the weight. The resulting vector is plotted as a spatial pattern to visualize the dominant spatial correlations of the background noise for each brain (FIG. 9). We note that quite generally, temporal MEG sensors are spatially correlated as denoted by a similar coloring of these zones. In control brains, the spatial structure is in general patchier than in ASD brains. To quantify these observations, we computed the spatial complexity of these patterns with an algorithm that was recently introduced in the literature. Briefly, this algorithm measures how well 2-dimensional interpolation can predict the value of the pattern at the position of a given sensor, given the values in the surrounding sensors (see Methods). The values of spatial complexity are significantly higher in control than in ASD (FIG. 9, inset; Wilcoxon ranksum test, p, 0.01). This demonstrates that spatial correlations are constrained to smaller patches on the brain in control subjects, whereas they extend over wide areas in ASD subjects, in other words, there is more variability in the spatial pattern of the background activity (or noise) in children without autism.

### Discussion

Our study has revealed alterations in brain connectivity and background noise in juvenile ASD patients, more specifically, signs of increased excitation were found from occipital to frontal areas. Perhaps even more interestingly, we found that background noise is spatially correlated over wide areas, that is, its spatial complexity is lower in ASD recordings. The analysis has been performed using a novel analytical method to investigate brain activity by determining two of its most fundamental aspects: the direction of functional connections and the temporal and spatial structure of the stochastic inputs driving cortical networks.

There is an increasing demand for adequate discrimination of patients in a variety of psychiatric syndromes using relatively safe and non-invasive methods such as EEG, MEG or neuroimaging recordings. The analytical techniques involved range from pattern recognition of neuroimaging data, as recently shown to classify patients with attention deficit hyperactivity disorder, to complexity measures derived from EEGs and graph theory using MEG data. These methodologies normally rely on quantification of activity, sometimes translated as “functional connectivity”, ignoring a most fundamental aspect of nervous system activity: that of the continuous presence of ongoing, background activity. As denoted by some investigators, sensory inputs act as modulators of the ongoing activity, and it is in this “noisy” background where intrinsic aspects of each nervous system can be found. Our analysis has the advantage of describing functional interactions through a deterministic component, the functional connectivity, and through a stochastic component, the background noise. We found even better discrimination using the latter, which according to the aforementioned comments should not come as a surprise. Of singular interest is the fact that the spatial variability of the noise is reduced in the recordings from ASD individuals. It is increasingly recognized that decreased variability in physiological signals is associated with disease, and specially a reduction of variability in brain signals seems associated with psychiatric and neurological conditions. Hence, our results support the view that cellular activities in brains should present a certain level of fluctuations in order to process information in the considered normal manners. Indeed, evidence for reduced fluctuations in brain signals associated with poorer behavioral performances has been provided recently. From a practical stance, the evaluation of spontaneous brain activity has been proposed as a biomarker in neuropsychiatric disorders and, from a more academic perspective, fluctuations in neurophysiological activity has been proposed to improve the exploration of the brain's dynamic repertoire.

The main connectivity change in ASD relative to control showing enhanced functional excitation from occipital to frontal areas is an indication of another general characteristic of aberrant brain function that is becoming apparent in current research: enhanced neural excitability seems to underlie neuropsychiatric disorders and has been proposed to be the basis for social dysfunction in general. In this regard, autistic-like symptoms in mutant mice is normalized by improving inhibitory neurotransmission using GABA agonists like clonazepam. Perhaps this tendency to show more excitability underlies the well-known epileptic co-morbidity in autism. Abnormal connectivity in autism has been described mostly based on neuroimaging (metabolic) data, as was mentioned in the introductory paragraphs above. In our study we find that fronto-occipital sensors display the major differences between the control and ASD group. Alterations of the frontal cortex have been noted in autism, and particularly an abnormal spatial organization in the microglial-neuronal components. Recent tensor imaging studies have also revealed white matter abnormalities in autism, in particular, a possible atypical lateralization in some white matter tracts of the brain and a possible atypical developmental trajectory of white matter microstructure in persons with ASD. Because our measures are derived from MEG signals, and thus detect local population activity mostly in the cortex, we speculate that the observed differences reflect a different activity in frontal cortical areas as these receive processed inputs from other cortical regions, specially sensory ones. Activity in sensory cortices is as essential as that of the normally more considered and studied higher-order association areas; for instance, in behaviors as diverse as the discrimination between free and forced actions, it is the activity at the sensors recording primary sensory cortices that best differential both actions. Because of the great importance of visual inputs, it is perhaps not surprising to see an alteration in occipito-frontal signals.

The term “functional connectivity” has been ambiguously employed to date. In some studies functional connectivity is synonymous with covariance, in some others with synchrony or coherence, etc. We propose here a concept of functional connectivity that has three important advantages: 1) Contrary to previous approaches, we do not focus on the analysis of functional connectivity in the context of psycophysical experiments but rather on ongoing, resting-state activity. This facilitates the estimation of functional connections because the activity of the underlying neural networks does not saturate, so the neural interactions can be well resolved. 2) Our method detects the direction of functional connections, i.e. whether area A excites (or inhibits) area B more strongly or vice versa. Other methods have been previously proposed to detect directionality of network interactions: a) Granger causality, b) the imaginary component of the coherency, and c) the coupling function of phase oscillators. However, methods a) and c) are model-dependent, i.e. they make assumptions about the nature of the signals that oftentimes do not apply to EEG/EMG recordings; and method b) is defined in the frequency domain, so its value depends on the frequency components of the signals. This limits its applicability as a measure of connectivity, which one wants to define by means of a number rather than as a function. 3) To our knowledge, our method is the only one to date that allows one to infer the temporal and spatial structure of the stochastic inputs driving the cortical networks in the brain's resting state. This is quite remarkable, as the classification of controls and ASD is even more accurate considering the spatial covariance of the noise, Q than using the connectivity matrix, W, indicating that noise in the brain is an important feature of the cognitive phenotype.

MEG recordings have some limitations to keep in mind. The signals detected by MEG and the source estimates derived from these signals reflect population-scale levels of activity in large neuronal networks. Every individual neuronal component from which an MEG signal is comprised possesses complex non-linear relationships with its synaptically connected neighbors and surrounding glia. The complexity of these interactions cannot be accessed with precise detail from the level of the MEG signal because it provides measurements that are too coarse to reveal such dependencies. As a result, insights gained from the investigation of MEG data are limited to coarse relationships between large populations of cells rather than the detailed understanding of interactions between individual cells. Moreover, spontaneous activity at any given sensor may contain activity from multiple distributed sources, and conversely, the activity of a single signal source can introduce coordinated changes at multiple sensors (cross-talk), which could lead to spurious interactions among MEG sensors. With these caveats in mind, all of our analyses focused on changes in one group (ASD) relative to the other (control). For example, we do not make any conclusions from the absolute connectivity between areas A and B in the brain, but rather from the change in connectivity between A and B in ASD compared to control.

To investigate the cross-talk between sensors we plotted the covariance between two channels as a function of their relative distance. The data points are from an individual in the control group but the same pattern is observed in all individuals from both groups. Clearly, for sensors that are less than 10 cm apart, the correlation coefficient between covariance and distance is negative and large in absolute value. There are two components contributing to this negative correlation. One is biological, meaning that anatomical connections are much more likely between nearby areas. This is consistent with previous animal studies showing that nearby neurons in the cortex display synchronized activities in vivo. The other is spurious, indicating cross-talk between sensors. These two components are mixed in the MEG setup and cannot be easily resolved. However, we can show that the trend is comparable between all individuals and indistinguishable between both groups. The distribution of correlation coefficients is statistically the same for control and ASD. This implies that as long as one focuses on changes in functional connectivity of one group relative to the other, the effects of any cross-talk between sensors should cancel out, and therefore, the results will not be contaminated by the limitations of the MEG setup.

Potential limitations in the design of the experiments cannot account for the differences between groups uncovered with our method either. To facilitate the participation of the children in the experiments and minimize distraction, they were asked to press a button at will with their right hand a few times during the recording session (30 s for each subject). Button pressing was not significantly different between both groups (p=1; Wilcoxon sum-rank test). Data preprocessing, in particular, the removal of a few principal components (PC) from the recordings to filter out eye-blinking and movement artifacts (see Methods) did not have differential effects either. Specifically, the number of removed principal components was not significantly different, as shown in Fig. S2 (p=0.83; Wilcoxon sum-rank test). The gender mismatch is also unlikely to account for differences between both groups. In control there were 6 males and 4 females, whereas in ASD there were 9 males and no females. In this regard we first note, that Asperger's syndrome is between 4 times and 12 times more frequent in males than females, so it is methodologically very challenging to have sex-matched groups. However, the significant differences that we observe between ASD and control cannot be attributed to a gender-ratio mismatch because the control group is very homogeneous: for example, there are no significant differences in the spatial complexity of the background noise between the boys and girls within the control group (p=0.76; Wilcoxon rank-sum test). In brief, considering that none of these parameters (button pressing, number of PC removed and gender specificity) were different between groups, it is very unlikely that they can account for the consistent differences in functional connectivity and spatial complexity that we observe between groups.

As in any population study, one must take into consideration the possibility of finite size effects. In statistical terms, the fact that we can establish significant differences in functional connectivity and background noise in relatively small populations suggests that those features are robust. Usually large sample sizes are required to establish the level of significance and accuracy that we obtained in our studies for a smaller sample size. We also note that all the participating children from the ASD group had been clinically diagnosed with Asperger's syndrome. They clearly had behavioral and cognitive differences with respect to children in the control group. ASD certainly develops over time, but once it is diagnosed based on cognitive parameters it should be possible to observe differences in terms of neural dynamics as well. And that is what we have addressed in this study.

There are two natural extensions of our work for future studies. The first extension is to investigate the applicability of our approach to other cognitive phenotypes to identify alterations in functional brain connectivity and background noise activity. The second extension is methodological and consists in considering nonlinearities in the stochastic model so that it can be applied beyond the resting state to investigate how functional connectivity is modulated by sensory stimulation, attention and other cognitive tasks.

### Methods

Participants and Magnetoencephalographic Recordings Data were drawn from a larger sample of children enrolled in a previous study. Data from nineteen children, 9 with Asperger's syndrome and 10 age-matched control children without any know neurological disorder, were analyzed. Age range was between 6 and 14 years for the controls (mean: 11.2 years; standard deviation: 2.6 years) and between 7 and 16 for ASD (mean: 10.8; standard deviation: 3.5). The 9 children with Asperger's syndrome were males while the 10 controls were 6 males and 4 females. This gender mismatch is due to the fact that Asperger's syndrome is between 4 times and 12 times more frequent in males than females, so it is methodologically very difficult to have sex-matched groups (see additional comments on gender mismatch in the Discussion). The children's parents provided written consent for the protocol approved by the Hospital for Sick Children Research Ethics Board. Participants met the criteria for ASD based on DSM-IV. Patients were evaluated by the psychologists in the Autism Research Unit of the Hospital for Sick Children or were recruited from the Geneva Centre for Autism and Autism Ontario.

Magnetoencephalographic (MEG) recordings were acquired at 625 Hz sampling rate, DC-100 Hz bandpass, third-order spatial gradient noise cancellation using a CTF Omega 151 channel whole head system (CTF Systems Inc., Port Coquitlam, Canada). Out of the 151 sensors, we discarded 10 that were not comparable across all patients due to artifacts or a very low signal-to-noise ratio. Our analysis thus focused on the recordings from the remaining 141 sensors in all patients. Subjects were tested supine inside the magnetically shielded room. Head movement was tracked by measuring the position of three head coils every 30 ms, located at the nasion, left and right ear, and movements less than 5 mm were considered acceptable. Children were instructed to remain at rest during the recording session that lasted between 30 and 60 s per child. To facilitate the involvement of the children in the experiment and minimize distraction, they were asked to press a button at will with their right hand a few times during the recording session. For each child, an epoch of 30 s was taken off for analysis of functional brain connectivity. All children were awake and had their eyes open during the experiment.

Eye-blinking and muscular artifacts were present in most recordings. These artifacts appeared across many channels with high amplitude relative to baseline fluctuations and thus dominated the first few principal components of the data. Removal of 1 to 6 principal components efficiently eliminated the artifacts without affecting the actual baseline fluctuations.

Obtaining W from Recordings of Brain Activity

Rewriting system (1) in vector notation one has

$$\frac{d \vec{x}}{d t} = W \vec{x} + \vec{η} ( t ) . \qquad ( 2 )$$

For a multivariate Ornstein-Uhlenbeck process like system (2) the time-lagged covariance, C(τ)≡ [symbol] \vec{x}(t+τ) \vec{x}(t)^{T} [symbol] where t is the lag and the brackets indicate a temporal average, satisfies

C(τ)=exp(Wτ)C(0),

where exp ( . . . ) is the exponential matrix function. For τ□1/∥W∥ with ∥ . . . ∥ indicating the matrix norm, one has

C(τ)≈(I+Wτ)C(0)

where I is the identity matrix. This allows us to obtain an expression for the connectivity matrix, W as a function of the lag, t

$$W = \frac{1}{τ} ( C ( τ ) {C ( 0 )}^{- 1} - I ) . \qquad ( 3 )$$

Then, to compute the empirical W, we choose t such that the trace of the covariance matrix of the residuals [symbol] \vec{η}(t) \vec{η}(t)^{t} [symbol] is minimal, which corresponds to minimizing the mean quadratic error of model (2). In our data, this is attained for the smallest possible value of t with a sampling frequency of 625 Hz, t˜1=625 Hz˜1:6 ms˜dt, where dt is the integration time step.

Finally, we note that if the zero-lag covariance matrix of the data, C(0), is not invertible, one may replace its inverse matrix, C(0)⁻¹ with its pseudo-inverse in equation (3).

### Theoretical and Empirical Q

For a multivariate Ornstein-Uhlenbeck process like (2), the covariance matrix of the residuals, Qis related to the covariance matrix of the signals, C(0) and the drift operator (connectivity matrix, W) via

Q_{T}=−½(C(0)W^{T}+WC(0))

where the subindex T refers to a theoretical calculation of Q. Obviously, the covariance matrix of the residuals can also be directly calculated from the data, which we refer to as empirical estimation with subindex E. To this end, one first computes the time derivatives of the signals, d\vec{x}(t)/dt and obtains the residuals as the difference \vec{η}(t)=d\vec{x}(t)/dt−Wx(t). Then, one computes their covariance matrix as

Q_{E}= [symbol] \vec{η}(t)\vec{η}(t)^{T}

[symbol]

The fact that QT and QEare practically identical, as shown in FIG. 6C, provides a strong validation of model (2).

### Support Vector Classifier

The vectors representing reshaped connectivity matrices (see FIG. 2A) are fed into the algorithm of the support vector classifier. The output of the algorithms returns a set of n “support vectors”, \vec{s}ᵢ, weights aᵢ, and bias b that are used to classify a given reshaped connectivity matrix, \vec{w} according to the following equation

$$c = {\sum }_{i = 1}^{n} {a}_{i} K ( {\vec{s}}_{i} , \vec{w} ) + b , \qquad ( 4 )$$

where K is a kernel function. In the case of a linear kernel, which is the one used here, it is the dot product: K(\vec{s}ᵢ, \vec{w})=\vec{s}ᵢ·\vec{e} and equation (4) defines a plane in the high-dimensional space of the reshaped matrices. If c≧0, then \vec{w} is classified as a member of group 1 (e.g. control), otherwise it is classified as a member group 2 (e.g. ASD). The results of the classification analysis are shown in FIG. 7. A similar analysis can be performed to classify the reshaped Q (FIG. 8).

### Group Separability and Cross-Validation Techniques

Group separability was addressed by comparing the performance of the support vector classifier with a linear kernel on the original groups to the performance of the same type of classifier on randomized groups (obtained by randomly permuting the group labels). In our specific implementation, the randomization was carried over 10,000 times. Classification performance was computed for both, the original groups and the randomizations, by leave-one-out cross-validation on every subject. A feature vector for each subject is made using the connectivity matrix W (size: 141×141) reshaped into a one-dimensional list of values, a vector of features of dimension 19,88161. The original Accuracy, Specificity, Sensitivity and F-Score of the classifier were then compared to those obtained for the randomizations. For each one of these four parameters, p-values were obtained by finding the average number of times that each parameter in the randomized population had equal or larger values than those of the classification from the original groups. This test provides a measure of the statistical significance of the classifier performance as well as general group differences. The results are shown in FIG. 7. The same procedure applied to the covariance matrix of the noise Q, produces the results shown in FIG. 8.

Random Permutation Test and z-Scores

To determine if a given element of the connectivity matrix, Wij is on average different in ASD from control, we applied the following random permutation test. We took the n=10 values from the control group and m=9 values from ASD group, as depicted in FIG. 3. To test whether the difference of the means in both groups was significantly different, we first gathered the n+m data points into one group and draw n points randomly to form a new group, allocating the remaining m data points to form a second group. This way we created a random surrogate data set from which the difference of the means was calculated. We then iterated this process 10,000 times to build a probability distribution of the difference of the means for surrogate data. If the difference of the means from the actual data was larger than the 99 percentile of this distribution, then that value was considered to be statistically significant with 99% confidence. The p-value was calculated as the integral of the distribution from left end (−∞) up to the actual value.

We note that the distribution of the difference of the means for the surrogate data converges fairly quickly to a Gaussian as the number of surrogate samples increases. This allows us to easily compute the z-score of the change in connectivity as the actual difference of the means divided by the standard deviation of this distribution.

### Spatial Complexity

Spatial complexity was calculated using a similar algorithm to that already described in. The spatial pattern was obtained as the weighted average of the principal components (eigenvectors of Q) according to their variance (eigenvalue). For each subject this procedure results in a vector X of 141 values which is then fed into the spatial complexity algorithm. The intuition behind the algorithm is to capture the heterogeneity of the spatial distribution of values. A pattern is considered to be spatially complex if it contains values at some spatial location (sensor position) that are badly predicted by the values of the neighboring sensors. The algorithm calculates the squared root of the mean squared difference of each value in X with the value predicted at the respective location by a smooth interpolation of its neighbor values using MATLAB griddata v4 method. Higher values of this algorithm correspond to more complex patterns.

In an embodiment, the systems and methods above are represented and redescribed below along with the methodologies used and results.

### Patient Selection and Exclusion Criteria

Clinical and electrophysiological data of all patients who underwent an intracranial EEG evaluation using SEEG electrodes between January 2009 and January 2012 were reviewed (n=149). Patients who had intractable TLE and underwent a standard anterior temporal lobectomy with one year follow-up were included in this study (n=23). All patients underwent appropriate pre-surgical evaluation with noninvasive video electroencephalography evaluation, high resolution brain MRI (Siemens 1.5 Tesla SP system, Erlangen, Germany) using a standardized epilepsy protocol, PET scan and detailed neuropsychology evaluation. The recommendation to proceed to an invasive evaluation was made during a multidisciplinary patient management conference, typically for localization of the ictal onset zone in patients with conflicting non-invasive data or to rule out pseudo-TLE. Multiple electrodes were placed using a robotic system (ROSA™, Medtech, Montpellier, France) according to a pre-implantation hypothesis about the possible epileptogenic zone. Each electrode had 10 cylindrical platinum contacts which were 2-3 mm long and 0.89 in diameter (Ad-tech, Racine, Wis., USA). A favorable outcome was defined as complete seizure-freedom one year after surgery. Patients who had topectomies, temporal pole resection or incomplete resection the anterior temporal lobe were not included to facilitate the comparison between patients.

### Data Acquisition and Preprocessing

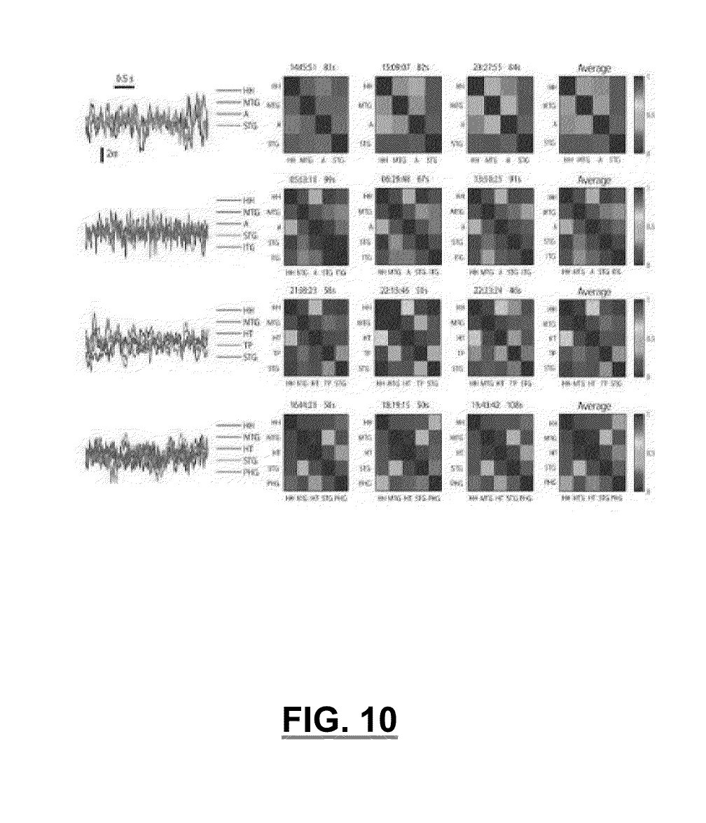

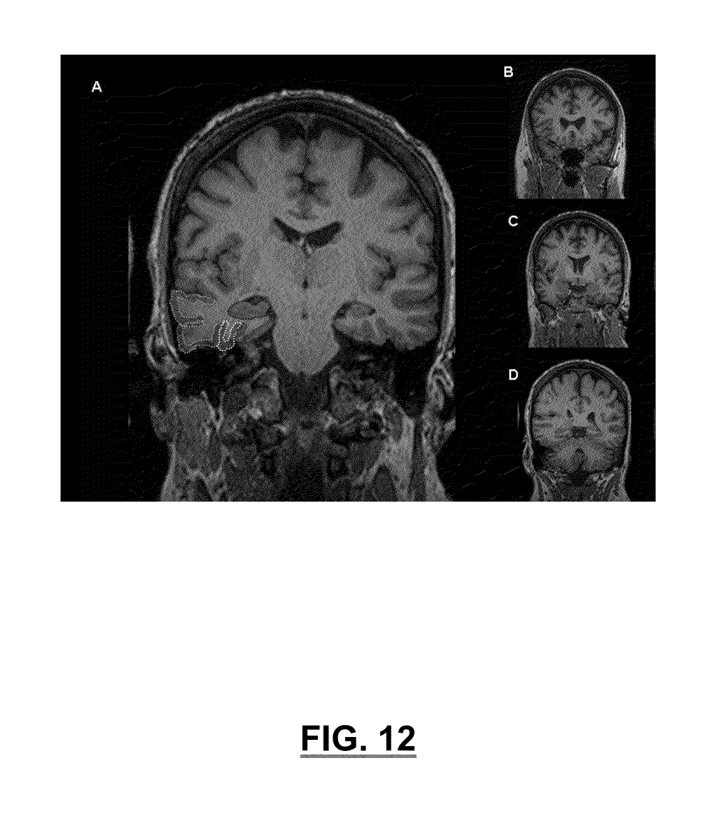

To identify the areas where electrode contacts were placed, the temporal lobe was first parcellated into 9 standardized regions, namely: hippocampus head (HH), hippocampus tail (HT), amygdala (A), superior temporal gyms (STG), medial temporal gyms (MTG), inferior temporal gyrus (ITG), fusiform gyms (FG), parahippocampal gyrus (PHG), and temporal pole (TP). Recordings were not obtained from all the 9 parcellated regions in the same patient due to lack of electrode contacts as the implantation schema differed slightly between patients depending on the results of the non-invasive evaluation. The location of the contacts was then compared visually to publicly available parcellation maps, allowing us to identify the areas from which the recordings were made in the temporal lobe. Parcellation of the temporal lobe was not automated. FIG. 12 shows the corresponding parcellated regions. SEEG traces were recorded at sampling rates of 1, 0.5, or 0.2 kHz depending on the recording and storage capabilities available when the patient was evaluated. The SEEG traces were off-line filtered with a custom-made digital filter in the frequency domain (pass band: 5 to 50 Hz; stop frequencies: 0 and 60 Hz). From the recordings of each patient, we selected three nonoverlapping segments of interictal activity of approximately 90 s; sometimes a bit shorter or longer so that intervals with repetitive interictal spikes were avoided in each segment. The segments were taken at different times over a time span of one to several hours (examples shown in FIG. 10). We selected samples prior to the first seizure to avoid sampling segments with high synchrony due to the ictus. Samples within 30 minutes of the seizure were not selected for analysis to avoid preictal changes in EEG. The state of the patient (awake, drowsy, sleep) and medication withdrawal were not taken into account during selection of SEEG segment for analysis. Our analysis was restricted to SEEG signals from the temporal lobe for consistency across patients, even though the anatomical locations of electrode placement also included the orbitofrontal region, insula, cingulate or precuneus. Spike-free (or almost free) segments were selected and any residual spikes were “clipped”. The difficulty in choosing an ideal reference is well known and various methods have been suggested to overcome it. We used the ‘0 V’ reference (effective ground) provided by our SEEG recording system (Nihon Kohden, Tokyo, Japan) which is a linked C3/C4 reference. Using simulation studies, Rappelsberger found that the most reliable results for evaluation of coherence were obtained when this referential montage was used compared to bipolar, common average or source derivation recording.

### Data Analysis and Functional Connectivity

Functional connectivity between two areas in which two electrode contacts were located was defined as the normalized covariance of their recorded traces. More generally, and quantitatively, consider a set of n simultaneous recordings of intracranial EEG activity, denoted by xᵢ(t) where the subindex refers to the i-th trace (i=1, 2, . . . , n), and t indicates the ordinal time stamp of the recordings (t=1, 2, . . . , T). The functional connectivity matrix between area i and area j is defined as Pearson's correlation coefficient between xᵢ(t) and xⱼ(t)

$${C}_{ij} = \frac{{\sum }_{t = 1}^{T} ( {x}_{i} ( t ) - {\overline{x}}_{i} ) ( {x}_{j} ( t ) - {\overline{x}}_{j} )}{\sqrt{{\sum }_{t = 1}^{T} {( {x}_{i} ( t ) - {\overline{x}}_{i} )}^{2}} \sqrt{{\sum }_{t = 1}^{T} {( {x}_{j} ( t ) - {\overline{x}}_{j} )}^{2}}} ,$$

where the mean value of

$${\overline{x}}_{i} = ( 1 / T ) {\sum }_{t = 1}^{T} {x}_{i} ( t ) .$$

Note that the values of all matrix elements are bounded between −1 and 1. Also, all diagonal elements are 1 by definition (Cᵢᵢ=Cⱼⱼ=1) and are irrelevant for our analysis.

### Support Vector Classifier

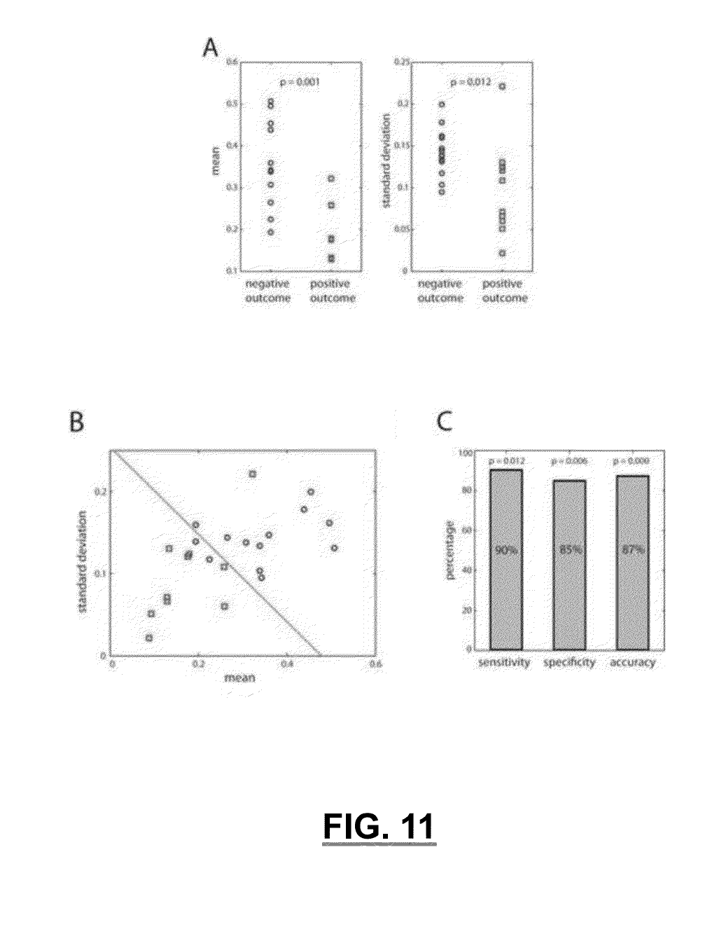

For each patient, we calculated the mean and standard deviation of the off-diagonal elements in the connectivity matrix. These two values respectively correspond to the x and y coordinates of each point in FIG. 11B. A support-vector classifier (SVC) is a standard algorithm to optimally separate two labeled datasets from each other. In the case of two dimensions, as in FIG. 11B, this translates into finding a line that separates the plane in two halves. If a perfect separation is possible, each half will contain data points of one label but not the other. When the separating line is constrained to be straight, as is the case in our study, the SVC is equivalent to a linear discriminant analysis. The two labels (categories) used in our analysis are whether the patient had a positive (seizure freedom) or negative (seizure recurrence) outcome after temporal lobectomy.

### Accuracy, Sensitivity and Specificity of the Classifier

SVCs have the advantage over clustering methods of tolerating some overlap (soft margin) between datasets. When overlap exists, the mere existence of a separating line is not very informative, since both groups are not 100% separable. In such cases, the classification performance index, also known as accuracy (see next section), is a better parameter to quantify the goodness of the discrimination, which is computed with the leave-one-out method. First, the separating line is computed after removing one point from the data set. Then, one tests if the point that was left out is correctly classified. These three steps (removal of a point, calculation of the discriminator and classification of the point removed according to that discriminator) are then iterated for all data points. The fraction of points that are correctly classified for each stimulus is the accuracy of the classifier. High accuracy means that the separating manifold is fairly insensitive to the removal of any given point, and hence robust to perturbations of the data set. This in turn implies that the space in which the points are represented is divided in two category specific halves, despite some overlap between datasets.

Using the leave-one-method, one can also compute the sensitivity and specificity of the SVC, which are computed from the number of true and false positives (TP and FP, respectively) and true and false negatives (TN and FN, respectively), which in our case are defined as: TP, surgical outcome for the patient that was left out prior to the calculation of the separating line is positive (seizure freedom) and the SVC predicts a positive outcome for that patient; FP, surgical outcome is negative (seizure recurrence) and the SVC predicts a positive outcome; TN, surgical outcome is negative and the SVC predicts a negative outcome; FN, surgical outcome is positive and the SVC predicts a negative outcome. One then has: sensitivity=#TP/(#TP+#FN), specificity=#TP/(#TN+#FP), accuracy=(#TP+#TN)/(#TP+#FP+#TN+#FN), where “#” means “number of”.

### Random Permutation Test (Cross-Validation of the SVC)

To determine whether the sensitivity, specificity and accuracy values of the SVC are statistically significant, we use a shuffling approach by randomizing the outcome of surgery, which is a binary variable (1: successful surgery; 0: unsuccessful surgery). Specifically, we take a random permutation of the 0's and 1's attributed to the 23 patients. For each randomization we recomputed the SVC, its sensitivity, specificity and accuracy using the leave-one-out method. We repeat the process for 10,000 randomizations and compute the probability (p-value) of obtaining a sensitivity, specificity and accuracy values equal to or higher than those obtained from the actual data. These p-values are reported in FIG. 11C.

### Results

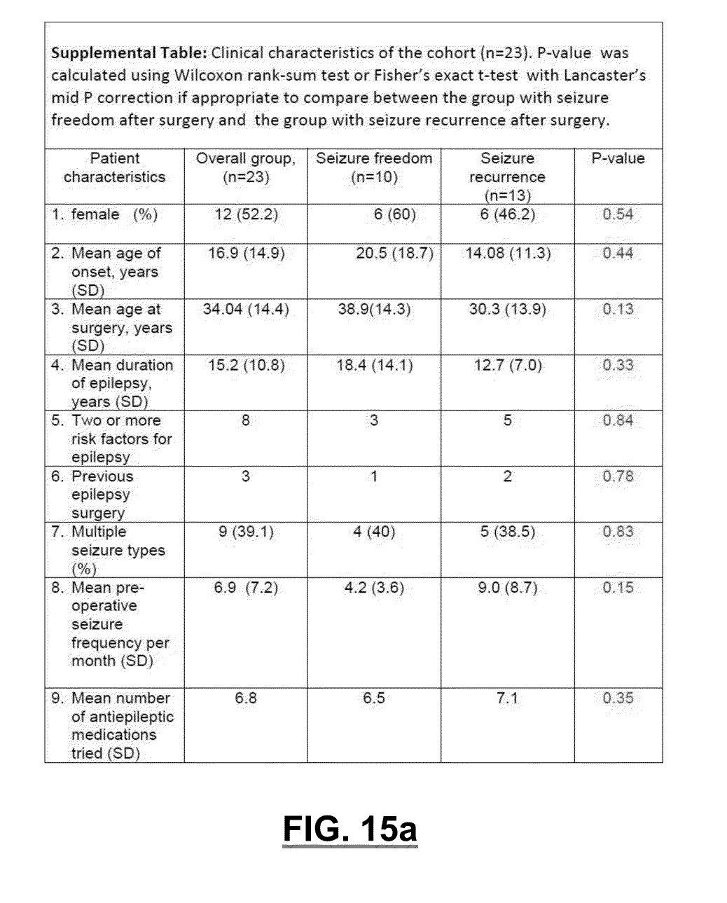

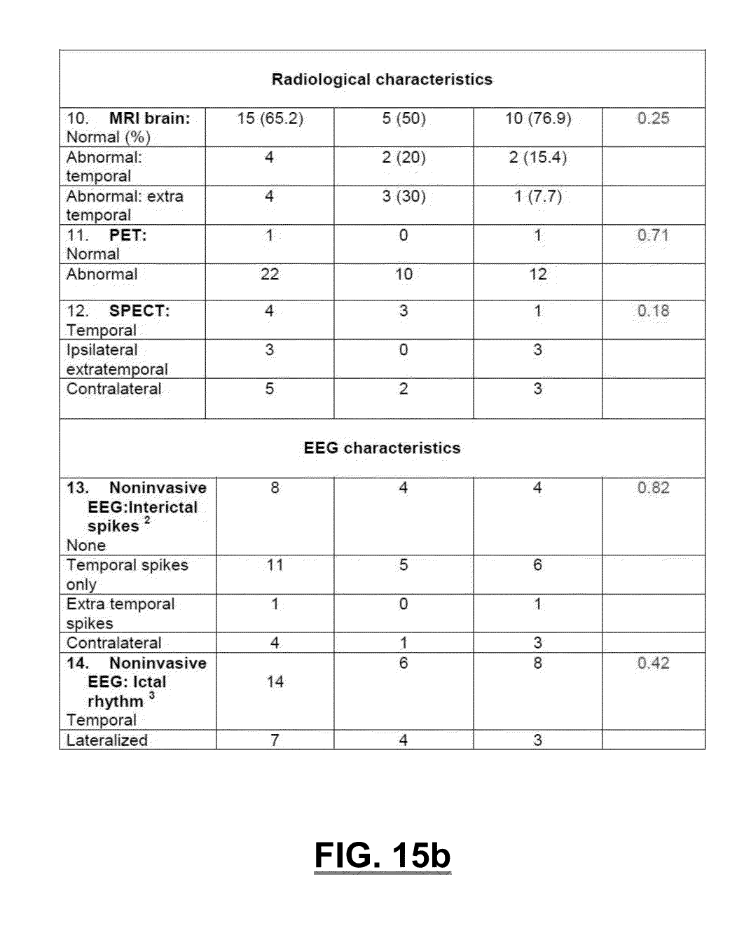

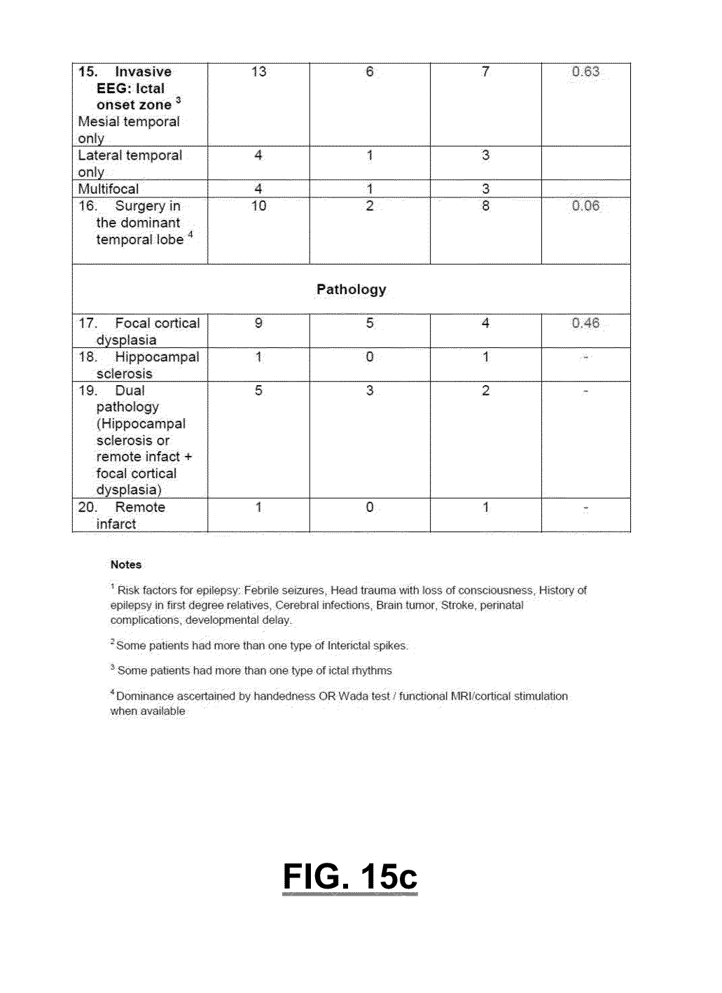

The clinical characteristics of all the 23 patients who underwent standard temporal lobectomy and were considered in our study are summarized in FIG. 15. Briefly, the patients' age ranged from 18 to 59 years and 48% were male. 65% of the patients had a normal MRI. Three patients had prior epilepsy surgery and 9 patients had multiple seizure types. Nine patients were found to have focal cortical dysplasia on pathology and five patients had hippocampal sclerosis in addition to focal cortical dysplasia. Only 43.48% remained seizure free at the end of one year of follow up. The high rate of seizure recurrence reflects the expected low rates of seizure-freedom in patients whose non-lesional, poorly localized, intractable epilepsy necessitates evaluation using invasive electrodes.

FIG. 10 shows representative four-second long traces of interictal activity from SEEG recordings (left) for four different patients (top to bottom). Traces in different colors represent records from different areas in the temporal lobe, as indicated. Even though these samples were arbitrarily chosen, the fluctuating activity clearly shows some level of entrainment between certain pairs within the same patient. This entrainment is quantified in the matrices plotted next to the right. The matrices display the pair-wise correlation coefficients computed over three nonoverlapping segments of the recordings starting at different times (indicated at the top, left in hours: minutes: seconds) with a duration of ca. 90 s (indicated at the top, right in seconds). The fourth matrix on each row is the average of the correlation matrices for the three segments in that patient. Overall the correlation matrices are fairly stable over time as they are consistent across different segments.

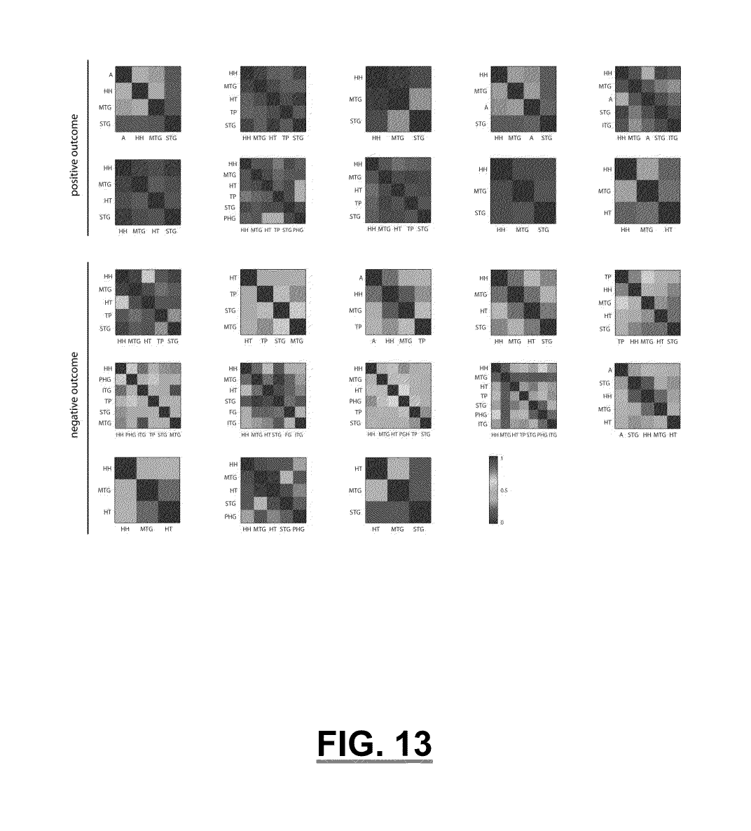

A detailed inspection of the averaged connectivity matrices for all patients reveals that, with few exemptions, patients who had a positive outcome after surgery had weaker connections and more homogeneous in strength (see FIG. 13). To quantify this observation, we computed the mean and standard deviation of the off-diagonal elements of the averaged connectivity matrices for each patient. FIG. 11A (left) shows the mean connectivity values for all patients with negative (left) and positive (outcome). Patients with a positive outcome tend to have smaller values of the mean connection strength. The difference of the medians is statistically significant (p=0.001; Wilcoxon sum-rank test). Similarly, FIG. 11A (right) shows the standard deviation of the connectivity values for all patients. The variability of the connection strength also tends to be smaller in patients with a positive outcome. Despite one outlier, the difference of the medians is also significant (p=0.012; Wilcoxon sum-rank test). When the mean connectivity and its standard deviation are considered at the same time, the separation between both groups of patients becomes even more apparent, as shown in FIG. 11B. A linear discriminator can separate the data points from each group with 3 misclassified patients out of 23 (13% error). We found that 90% of the patients who had weak and homogenous connections were seizure free, whereas 85% of the patients o had stronger and more heterogeneous connections within the temporal lobe had recurrence of seizures. Using a cross-validation test based on the leave-one-out method and random permutations (see Methods), we computed the sensitivity, specificity and accuracy of the linear discriminator, which yields the results shown in FIG. 11C. We note that none of the 20 parameters shown in FIG. 15 summarizing the patients' clinical data is significantly different between both groups (all p-values are larger than 0.05). This implies that functional connectivity, as defined here, adds an important prognostic value to currently used parameters in clinical practice.

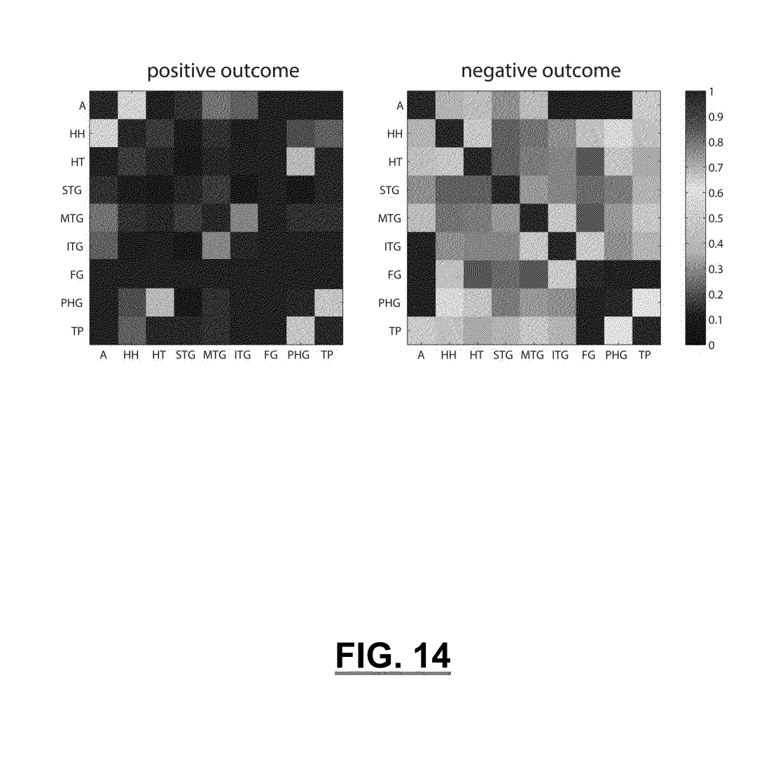

Finally, we investigated if the functional connectivity between specific pairs averaged across patients correlated with the surgical outcome. FIG. 14 shows the averaged connectivity values between areas for the positive (left) and negative (right) outcomes. It is again evident that a positive outcome is overall associated with low connectivity values except for the interaction between the hippocampus and amygdala which is on average above 0.5. High connectivity values are in general associated with a negative outcome, especially between the parahippocampal gyms and the temporal pole. Entries shaded in black in the connectivity matrices correspond to pairs that were not present in our pilot study. A larger dataset would be necessary to evaluate if the functional connectivity between specific pairs also has a predictive value for the outcome of temporal lobe epilepsy.

### Discussion

Our study shows that the matrices of functional connectivity have a predictive power for the outcome of epileptic surgery. Our study showed that 90% of the patients who had weak and homogenous connections were seizure free, whereas 85% of the patients who had stronger and more heterogeneous connections within the temporal lobe had recurrence of seizures In particular, by looking at the mean and standard deviation of the functional connections it is possible to predict the outcome with 87% accuracy. This is quite remarkable considering that our analysis is based on two assumptions: 1) there is only one epileptogenic zone in the brain that is located in the temporal lobe; 2) the electrodes properly sampled that zone or its immediate neighbors. The main reason for the prediction error is probably due to the existence of more than one epileptogenic zone in our patient population, which explains the high seizure recurrence rate. Even so, the specificity and accuracy of our predictions are well above the 99% confidence level and the sensitivity close below, but well above the 95% confidence level.

In previous studies, localized areas of synchrony were recorded in the seizure onset zone and removal of these regions improved seizure outcome. These were recorded using grids with electrode contacts which were relatively close (approximately 1 cm apart) pointing to the fact that these discrete regions of synchrony in the seizure onset zone are highly localized. In fact, Ortega and collaborators observed that surgical resection of highly localized regions of synchrony detected in electrocorticography predicted better outcomes with epilepsy surgery, but surgical resection of broadly distributed regions of synchronization clusters did not. Similarly, in recordings from intracranial EEG electrodes placed in various temporal lobe structures, we found heterogeneous functional connectivity between widely separated regions in the temporal lobe in patients who had recurrent seizures after surgery. We also saw high ynchrony (correlation) in one patient who was seizure free after temporal lobectomy but was falsely predicted to have recurrent seizures. It is possible that in this patient we recorded SEEG signals from one of the hyper-synchronous clusters reported by Ortega et al., due to a wider seizure onset zone or that the electrodes were closer together compared to other patients.

We defined a favorable outcome as complete seizure freedom and any recurrence of seizures were classified as an unfavorable outcome. We did not classify outcome further into more subgroups because of low sample size

Two patients who continued to have seizures after temporal lobectomy had higher values of connectivity. We speculate that the seizure onset zone in those patients extended outside the temporal lobe and was not removed by a standard anterior temporal lobectomy. It is also possible that these two patients had multifocal epilepsy. Thus our analysis showed increased connectivity in the temporal lobe even though they had a poor surgical outcome.

Previous studies reported increased connectivity in the epileptic brain as analyzed from intracranial EEG signals. Some of those studies analyzed connectivity within the seizure onset zone. They also varied widely in the method of analysis of connectivity, the anatomical proximity of the regions of interest, the control group selected, the number of subjects and the measure used to correlate with seizure outcome. For example, Bettus and colleagues analyzed the nonlinear correlation values in intracranial EEG averaged over six different combinations in 4 different structures (anterior and posterior hippocampus, entorhinal cortex and amygdala) and compared it between patients with TLE and extra temporal epilepsy [34]. Fluctuations in functional connectivity in the early years of epilepsy may be another reason for this apparent contradiction.

Recent studies with MEG have reported a focal increase in coherence in the epileptogenic zone in patients with intractable TLE. In contrast, most of the fMRI studies found lower connectivity in the epileptogenic side in TLE. Pittau et al showed that in mesial TLE, the amygdala and the hippocampus showed decreased functional connectivity with wide spread brain regions including the default mode network, ventromesial limbic prefrontal regions, contralateral mesial limbic structures and pons. But focal regions of increased coupling were also noted in fMRI studies on 5 patients with intractable epilepsy. A recent fMRI study using Granger causality, found altered causal relationship between the epileptogenic zone and other cortical networks. The disparity of results obtained with different recording techniques is not surprising considering that EEG, MEG and fMRI detect different physiologic signals with different spatial and temporal scales. Understanding these discrepancies is an important challenge for future studies.

All our patients had a standard procedure with consistent amount and location of tissue removed. This means that even though the seizure onset zone, as concluded from invasive and noninvasive evaluation was limited to one temporal lobe structure like the amygdala or the hippocampus, a standard temporal lobectomy was performed. We tried to analyze the mean connectivity between different structures in the diseased temporal lobe which not only included the seizure onset zone but also a large area around it. This essentially allowed us to calculate the strength of the connections of the seizure onset zone with the surrounding region which may be diseased, but not necessarily generating seizures and also the connectivity between these surrounding areas.

The patient population that we selected was complicated enough to undergo invasive evaluation and the results may not be applicable to many patients with mesial temporal sclerosis who undergo surgery without an invasive evaluation. We feel that it is this specific group of patients with complicated temporal epilepsy who needs better measures to predict outcome. We tried to limit the effect of the type of epilepsy on the network dynamics by only selecting patients with TLE. But TLE is heterogeneous and its effect on network connectivity can vary, which we did not account for.

How does our study differ from the previous studies measuring connectivity and surgical outcome in epilepsy? In addition to the strength of connections which has been studied extensively, we applied a measure of variability of connectivity in our calculations which has not been used thus far to predict surgical outcome in epilepsy. We show that adding standard deviation, a simple measure of variability, to the mean of the correlation coefficient improves predictability of outcome. Analysis of loss of neuronal complexity has been used to lateralize unilateral TLE and to predict outcome in neocortical lesional epilepsy. Loss of variability and increased excitability was recently noted in virtual brains modeled to simulate pathological states like epilepsy. The correlation matrices of the patients give a strikingly visual representation of decrease in strength and loss of heterogeneity of connectivity in patients with a good outcome (FIG. 13).

The results thus demonstrate the promising potential of a correlation-based measure of functional connectivity from intracranial EEG to predict surgical outcomes after TLE surgery. Prospective studies with a larger patient sample are needed to determine further applicability of this measure as a diagnostic tool.

Although the embodiments of the present invention have been illustrated in the accompanying drawings and described in the foregoing detailed description, it is to be understood that the present invention is not to be limited to just the embodiments disclosed, but that the invention described herein is capable of numerous rearrangements, modifications and substitutions without departing from the scope of the claims hereafter. The claims as follows are intended to include all modifications and alterations insofar as they come within the scope of the claims or the equivalent thereof.

Having thus described the invention, we claim:

1. A method of determining functional brain connectivity and its application as a biomarker comprising:
measuring brain activity signals to detect a functional connection between a first region of a brain and a second region of said brain;
determining the direction of said functional connection by determining whether said first region excites or inhibits said second region more strongly than said second region excites or inhibits said first region;
using said functional connectivity as a biomarker to predict the existence of a condition;
wherein said functional connection is measured while said brain is in a resting state.

2. The method of claim 1, wherein said condition predicted by said biomarker is autism.

3. The method of claim 2, wherein said prediction is 94% accurate.

4. The method of claim 1, wherein said condition predicted by said biomarker is the presence of epilepsy in a lobe of said brain.

5. The method of claim 1 further comprising determining an epileptic zone based on said directional connectivity between said first region and said second region.

6. The method of claim 1 further comprising determining whether said first region is functionally isolated from areas surrounding said first region.

7. The method of claim 6, further comprising using said functional isolation of said first region as a biomarker to determine the existence of a condition.

8. The method of claim 7, wherein said condition is autism.

9. The method of claim 7, wherein said condition is the presence of epilepsy in a lobe of said brain.

10. The method of claim 1, wherein said measuring is performed using at least one of scalp EEG, invasive stereo EEG, intracranial EEG, MEG, and fMRI.

11. The method of claim 1 further comprising inferring a temporal and spatial structure of inputs driving the cortical networks of said brain in a resting state.

12. The method of claim 1, wherein the method is performed prior to a lobectomy procedure to determine the location an epileptic zone.

13. The method of claim 1, wherein the method is performed prior to lobectomy procedure to predict the success of said procedure.

14. The method of claim 12 wherein the accuracy of said prediction is 87%.
